(19) United States
(12) Patent Application Publication
(10) Pub. No.: US 2021/0057049 A1
(43) Pub. Date: Feb. 25, 2021

(54) Method in Bioprocess System

(71) Applicant: Cytiva BioProcess R&D AB, Uppsala (SE)

(72) Inventors: Gunnar Malmquist, Uppsala (SE); Per Liden, Uppsala (SE)

(21) Appl. No.: 17/045,775

(22) PCT Filed: Apr. 24, 2019
(86) PCT No.: PCT/EP2019/060429

§ 371 Date: Oct. 7, 2020

(30) Foreign Application Priority Data
Apr. 25, 2018 (GB) 1806752.0

(51) Int. Cl. G16C 20/30; G01N 30/88

(52) U.S. Cl. CPC G16C 20/30; G01N 2030/8804; G01N 2030/889; G01N 30/88

(57) ABSTRACT

The present invention relates to a method for estimating performance of a bioprocess material when used in a bioprocess system. The bioprocess material comprising at least two ingredients, each having data properties. The method comprises: i) obtaining (81) the data properties for the at least two ingredients used to produce the bioprocess material; ii) defining (82) procedures to process the at least two ingredients; iii) processing (83) the at least two ingredients according to the defined process parameters to obtain at least a product; iv) measuring (84) data properties of each product, v) calculating (85) data properties of each product based on the measured data properties of each product and/obtain or data properties from the at least two ingredients, and vi)if the product is the bioprocess material, processing (88) the measured and calculated data properties to estimate the impact of the bioprocess material on a target product in the bioprocess system, or vii) if the product is not the bioprocess material, treating the product as an intermediate material and repeating (87) steps iii)-vi) with each product as one of the at least two ingredients.

### TECHNICAL FIELD

The present invention relates to methods in bioprocess systems, e.g. chromatography systems and cell culture systems.

### BACKGROUND

Bioprocess systems, such as chromatography systems and cell culture systems, base their process on raw materials normally provided from an external provider. Examples of raw material are chromatography resins and cell culture media. The quality of the raw materials will affect the bioprocess and a significant part of all manufacturing investigations are related to raw material variability.

Furthermore, when preparing a column for a chromatography system, material from different lots, although within the specifications, may be mixed and thus, the performance of the resin in the column will be different compared with when material from a single supplier lot is used.

Variations in performance is undesirable because the overarching objective in bioprocess manufacturing is to maintain the manufacturing process in control.

Thus, there is a need to introduce a process for reducing the impact of variations in raw material characteristics.

### SUMMARY

An object of the present disclosure is to provide methods and devices configured to execute methods and computer programs, which seek to mitigate, alleviate, or eliminate one or more of the above-identified deficiencies in the art and disadvantages singly or in any combination.

The object is achieved by a method for estimating performance of a bioprocess material when used in a bioprocess system. The bioprocess material comprises at least two ingredients, each having data properties. The method comprises: obtaining the data properties for the at least two ingredients used to produce the bioprocess material; defining procedures to process the at least two ingredients; processing the at least two ingredients according to the defined process parameters to obtain at least one product; measuring data properties of each product; calculating data properties of each product based on the measured data properties of each product and/or data properties from the at least two ingredients; and if the product is the bioprocess material, processing the measured and calculated data properties to estimate the impact of the bioprocess material on a target product in the bioprocess system; or if the product is not the bioprocess material, treating the product as an intermediate material and repeating steps: processing the at least two ingredients; measuring and calculating data properties of each product, and determining if the product is the bioprocess material.

An advantage is that non-measurable characteristics of the bioprocess material are identified which may improve the product quality and/or process performance when used in a bioprocess system.

Further objects and advantages may be obtained from the detailed description by a person skilled in the art.

### BRIEF DESCRIPTION OF THE DRAWINGS

FIG. 1 illustrates a bioprocess system suitable for implementing the disclosed methods.

FIG. 2 illustrates a process for producing a raw material from ingredients.

FIG. 3 illustrates a unit operation used to illustrate a generic bioprocess flow.

FIG. 4 illustrates a generic bioprocess flow based on unit operations described in FIG. 3.

FIGS. 5a and 5b illustrate an example of the connection between raw material production and data properties.

FIG. 6 illustrates the effect of controlling process parameters based on the performance of the raw material used in the bioprocess system.

FIG. 7 illustrates in-process mixing between batches

FIG. 8 is a flowchart illustrating a process of estimating performance in a material intended to be used in a bioprocess.

FIG. 9 is a flow chart illustrating a process of adapting process parameters based variations in process parameters.

### DETAILED DESCRIPTION

Definition of Some Terms Used in the Description

When Producing Raw Materials for Bioprocesses, Also Called Bioprocess Material
Ingredients—are the materials used when manufacturing a raw material
Substances—An ingredient may comprise one or more substances
Intermediate materials—comprises treated/processed ingredients and is not the final product. Intermediate products are not always present since raw material may be directly produced from ingredients.
Raw material—comprises processed intermediate materials and is the final product that is used in a bioprocess.

When Using Raw Materials in a Bioprocess
Raw material—is the bioprocess material that is used in a bioprocess.
Process intermediates—the produced material after each unit operation (as described in more detail below).
Target product—product in a bioprocess.

A bioprocess may comprise several different steps, such as bioreactor to grow cells and produce the target product, filtering to produce feed material, chromatography to purify a target product available in the feed material, etc. Some of these steps require different raw materials (such as feed medium, chromatography resin, etc.) to be able to deliver the required output. Any variations in material properties can have an impact on the process and it is therefore necessary to have strict specification limits within which different parameters associated with a material may vary. Examples of such parameters are:
size distribution of particles
concentrations
ratios between different concentrations of substances in the material
functional characteristics

Although raw materials have their parameters within the specified intervals, mixing of different batches/lots without accounting for the material properties of the mixture may result in an undesired drop in product quality and process performance. The methods of the invention are particularly suitable for processes/systems where at least two ingredients or intermediate materials are different batches of the same material and where these batches may differ from each other with respect to one or more data properties, e.g. morphological data properties.

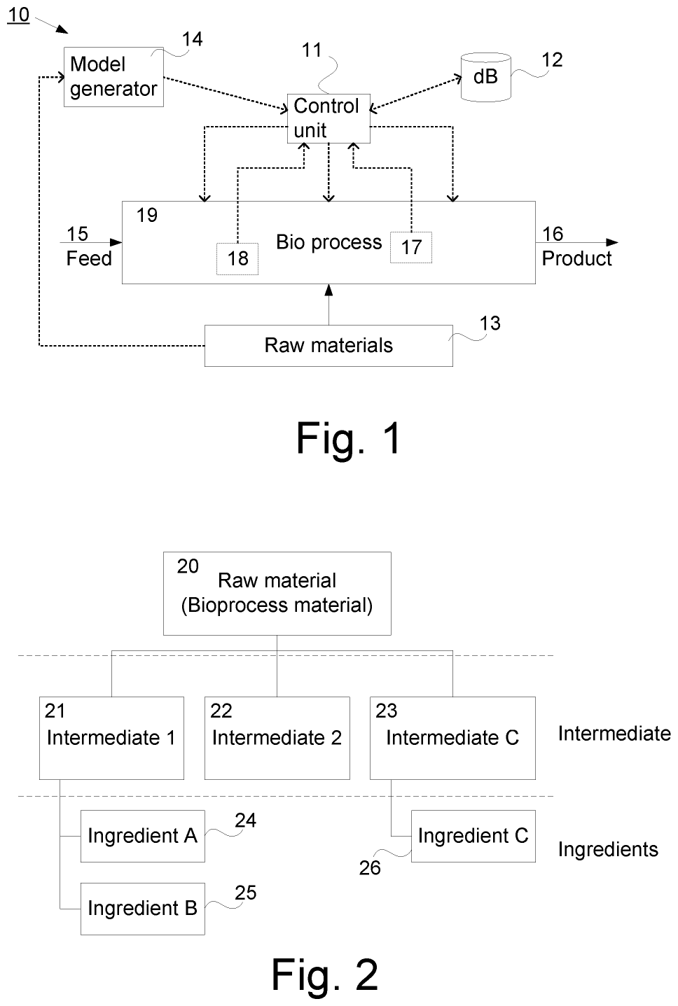

FIG. 1 illustrates a bioprocess system 10 suitable for implementing the disclosed methods. The system 10 comprises in this example one bioprocess 19, which is controlled by a control unit, or controller, 11. The controller has access to a data storage (in this example illustrated by a database 12), which may be locally arranged or implemented externally, e.g. in a cloud implementation. The bioprocess requires raw material 13 as illustrated by the arrow to produce a product (intermediate or target product) 16 from a feed 15. The feed 15 may be an intermediate from another bioprocess system (not shown). The controller 11 controls the process based on the data accessible from the database 12, which may contain historic data from previous runs, recipes, production process description, etc.

The raw materials 13 also contain a detailed description of the data properties associated with the respective raw material, as described in more detail below. The data properties (both measured and calculated) are provided to a model generator 14, where the impact of each raw material on the product 16 is estimated. This information is used by the controller 11 to adapt the process parameters to ensure product quality and/or process performance.

The data properties associated with ingredients, intermediates and products can comprise morphological data properties, i.e. properties representing physical structures. For particle materials, this can e.g. be particle size distributions (volume-weighted, number-weighted, entire distributions, distribution means, distribution widths, etc), particle shape (shape factor, sphericity etc.), distribution of substances within particles (e.g. distribution of magnetic material within magnetic adsorbent beads, distribution of high density matrials within expanded bed adsorption beads etc. For porous materials (including porous particles), morphological data properties can e.g. represent total porosity, pore size distributions, porous network structure, tortuosity, inverse size exclusion chromatography data (accessible pore volume vs. probe molecule size), etc.

The data properties can also comprise chemical composition data properties. These can e.g. be spectroscopic data, titration data, analytical chromatography data, elemental analysis data, amino acid composition data etc.

A third type of data properties is functional data properties. These are derived from functional testing of ingredients, intermediates or products and non-limiting examples of such data properties can be pressure-flow performance of packed bed columns, static or dynamic binding capacities of chromatography resins, dissolution rates of powders, cultivation performance of cell culture media etc. Functional data properties may correlate with morphological and/or chemical composition data properties. Often, the correlations with morphological data properties are more complex (e.g. non-linear), emphasizing the needs of applying the methods of the invention, particularly when mixing different batches/lots of a material.

An optional feature is sensors 17, 18, which measure selected process parameters, and are used to monitor the process.

An example of a bioprocess is a continuous chromatography, which is designed for purification of target products (such as proteins, biomolecules from cell culture/fermentation, natural extracts) in continuous downstream processes, e.g. using periodic counter current chromatography. The technology employs three or four chromatography columns to create a continuous purification step. The columns are switched between loading and non-loading steps, such as wash and elution. Continuous chromatography supports process intensification by reducing footprint and improving productivity. In addition, continuous chromatography is especially suited for purification of unstable molecules, as the short process time helps to ensure stability of the target product.

Another example of a bioprocess is a batch type chromatography having only one chromatography column, wherein the column sequentially performs loading and non-loading steps, such as wash and elution.

Still another example of a bioprocess is cell culture, wherein cells are grown in a bioreactor under the influence of cell culture media provided as raw material.

FIG. 2 illustrates a process for producing a raw material 20 from ingredients 24-26, as a bill of material. Ingredients are normally characterized and delivered with a certificate (Certificate of Analysis CofA) disclosing parameters associated with the ingredient lot such as substances and their concentrations, ratio between substances, particle size distribution, functional characteristics etc.

The raw material 20 is provided with data for use with the bioprocess control strategy, which is needed when controlling a bioprocess, even if this data is not measured at the raw material level. At the intermediate level, potential critical material attributes are measured and at the ingredients level, the ingredients 24-26 are characterized. However, properties of intermediates 21-23 and ingredients 24-26 cannot be propagated upwards without taking lot mixes into account. It should be emphasized that a CofA of the raw material 20 may not provide all the information necessary to fully characterize the material. Measurements and calculation on the intermediate material are required to be able to track the cause for an undesired behaviour of the raw material.

FIG. 2 also illustrates a method for manufacturing a bioprocess material 20 for a bioprocess, the bioprocess material comprising at least two ingredients 24-26, each having data properties. The method comprises processing the at least two ingredients 24-26 using predefined process parameters to produce at least two intermediate materials 21-23, obtaining data properties of each intermediate material, repeatedly processing the at least two intermediate material to obtain the bioprocess material 20, and identifying data properties of the ingredients and the intermediate materials relevant for the performance of the bioprocess material.

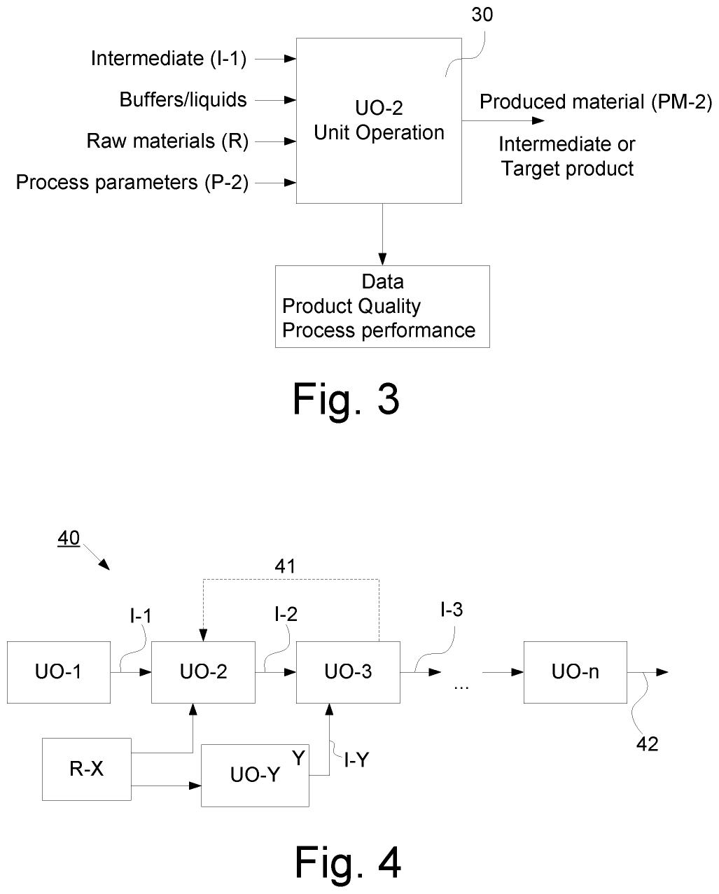

FIG. 3 illustrates a unit operation UO-2 30 used to illustrate a generic bioprocess flow 40. Intermediate I-1 (result from an earlier step in the bioprocess system) is fed into UO-2 together with raw materials R and buffers/liquids. Process parameters P-2 determine the operation of UO-2 to deliver the produced material PM-2, which may be an intermediate or the target product of the bioprocess system. Furthermore, the sensors of UO monitors and measures process parameters to estimate the product quality of the intermediate and/or target product. Alternatively, the UO estimates the process performance to optimize the process.

Data related to product quality (such as purity or host cell protein impurity level) and/or related to process performance (such as yield) is provided from the UO or some other data source to a controller, which may use the information to adjust the process parameters in order to compensate for undesired deviations in product quality and/or process performance. Quality attributes are measured on “in-process” materials. Each step, corresponding to the functionality of the UO, has its particular critical quality attributes CQAs. The CQAs are valuable for trending analysis. Performance attributes are typically yield, volume, etc.

FIG. 4 illustrates a generic bioprocess flow 40 based on several connected unit operations 30 described in connection with FIG. 3. This is a simplified illustration of a rather complex process model. A directed (and in most cases acyclic) process flow is shown, which in special cases may include cyclic paths (as illustrated by the dashed arrow 41 between UO-3 and UO-2). For analysis purposes, outputs of previous steps (upstream steps) are considered inputs to downstream steps, as explained in connection with FIG. 3.

UO-1 does not have any input, and is for example a working cell bank (vial). Inoculates are the output from UO-1 and is introduced into UO-2 together with raw material R-X. UO-2 may be a seed bioreactor and/or production bioreactor depending on the purpose of the UO and process parameters is provided as input to UO-2. Raw material R-X is also provided to UO-Y in which an intermediate I-Y is produced. Intermediate I-Y and the output from UO-2 are provided to UO-3, wherein I-3 is produced. Data regarding product quality and/or process performance are provided in each step and a controller (not shown) receives the data for controlling the process flow. The target product 42 is provided as output from the last UO-n.

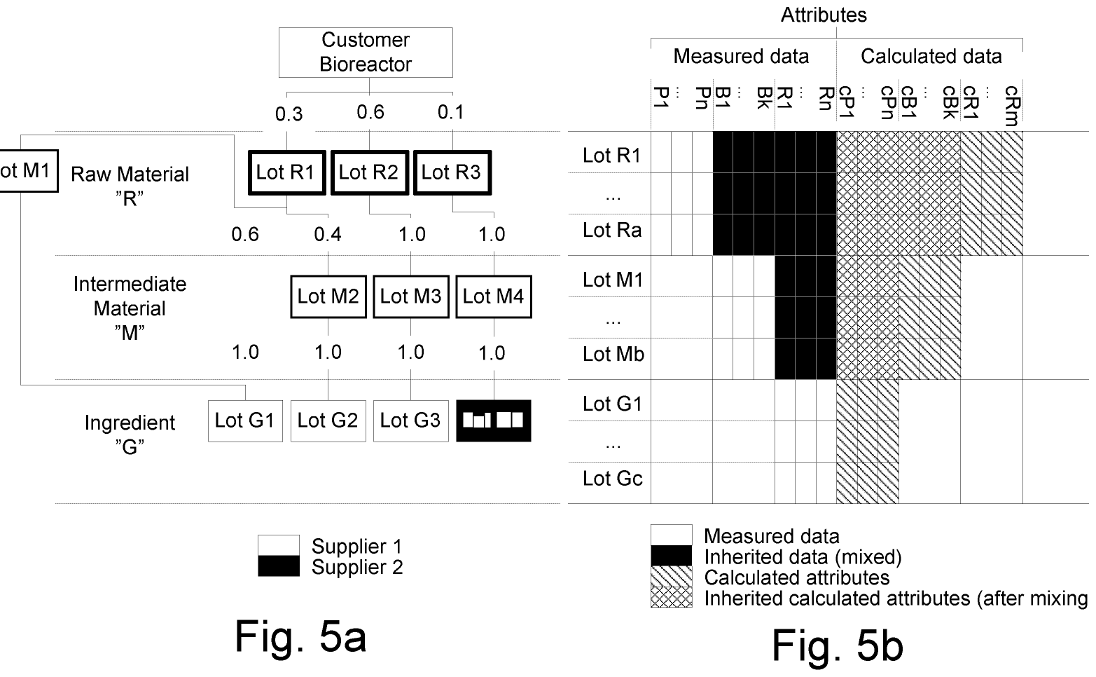

FIGS. 5a and 5b illustrate an example of the connection between raw material production and data properties. In this example, ingredients from four different lots and two suppliers are provided. Lots G1-G3 are from supplier 1 and Lot G4 are from supplier 2. Relevant data properties are measured, R1 . . . Rn for Lot G1 . . . Gc, etc. and relevant data properties are calculated, cR1 . . . cRm for Lot G1 . . . Gc. These attributes are inherited to the next level with intermediate materials, denoted Lot M1-M4. The mixing ratio between the levels are indicated in FIG. 5a.

Some of the mixing ratios are in this example 1.0, which means that no mixing is performed from ingredient to intermediate material. However, the ingredient may be subject to different treatments, such as washing, sieving, grinding, diluting, etc. which change the characteristics of the material compared with the characteristics of the ingredients. Relevant data properties are measured, B1 . . . Bk for Lot M1 . . . Mb, etc. and relevant data properties are calculated, cB1 . . . cBk for Lot M1 . . . Mb.

The first lot of raw material, denoted R1, comprises in this example material from two intermediate material lots M1 and M2, with a mixing ratio of 60% M1 and 40% M2 both having ingredients from Supplier 1. The second lot of raw material, denoted R2, comprises 100% of lot M3 having ingredient from Supplier 1, and the third lot of raw material, denoted R3, comprises 100% of lot M4 having ingredient from Supplier 2.

Before using the raw material in a bioreactor, the user mixes all three lots of raw material 30%, 60% and 10% to get the right amount of raw material for the bioreactor. The measured and calculated attributes illustrated in FIG. 5b assist the end user to determine the characteristics of the mixed raw material and to find the cause of a potential undesired behaviour in the bioprocess.

As said before, this is not a straightforward averaging, since it is a tree with performance parameters that are inherited during the processing steps. To illustrate this, a lot blending example of intermediate material lots is provided.

### Example 1

### Mixing Lots of Intermediate Material

Assume that there is two Bulk powder lots BP1 and BP2, both comprising ingredients A and B. However, the concentration of the respective ingredient is different in the Bulk powder lots, see table 1 below:

TABLE 1

| Bulk powder lot | Concentration [A] | Concentration [B] | Ratio [A]/[B] |
|---|---|---|---|
| BP1 | 20 | 100 | 0.2 |
| BP2 | 40 | 50 | 0.8 |

A Cell culture Liquid medium CCM is mixed by using 300 kg of BP1 and 600 kg of BP2.

If only the concentration ratio [A]/[B] is used to calculate the ratio of [A]/[B] in CCM, the volume weighted average will be 0.6, based on the following calculations:

$$\frac{300 kg}{900 kg} \cdot 0.2 + \frac{600 kg}{900 kg} \cdot 0.8 = 0.6$$

However, this is not the true ratio, since the knowledge of the actual concentrations in the different bulk powder lots will result in the true ratio of [A]/[B] in CCM:

$$Conc . Ingredient A \text{:} \frac{300 kg}{900 kg} \cdot 20 + \frac{600 kg}{900 kg} \cdot 40 = \frac{100}{3}$$

$$Conc . Ingredient B \text{:} \frac{300 kg}{900 kg} \cdot 100 + \frac{600 kg}{900 kg} \cdot 50 = \frac{200}{3}$$

$$Ratio [ A ] \text{/} [ B ] \text{:} \frac{100}{3} \cdot \frac{3}{200} = 0.5$$

Thus, the disclosure provides a genealogy traceability engine including mixed intermediate lots. It also provides calculation of attributes for lots mixed by the end user, as illustrated in connection with FIGS. 5a and 5b.

### Example 2

### Chromatography Resin

Another example relates to chromatography resin data, which illustrates the importance of providing inherited data from the ingredient when manufacturing the raw material (resin) for the chromatography system.

The resin is in this example Phenyl Sepharose™ 6FF HS and is normally provided with a Certificate of Analysis CofA comprising:
Ligand density
Lysozyme retention
RNase A retention

The resin is produced from one or more lots of Base Matrix (intermediate material) called Sepharose™ 6 Fast Flow base matrix. For each Base Matrix relevant parameters are measured during manufacture, such as:
Particle Size Distribution (PSD)
Porosity
Flow rate

These relevant parameters are provided as inherited attributes for the resin. Furthermore, the base matrix is manufactured from an ingredient Agarose with physicochemical properties that are also inherited via the base matrix to the resin.

This type of data properties are vital when investigating root causes to production issues, such as yield variation, but may also be used to adjust the process parameters to compensate for deviations from a predetermined process, i.e. a standard process.

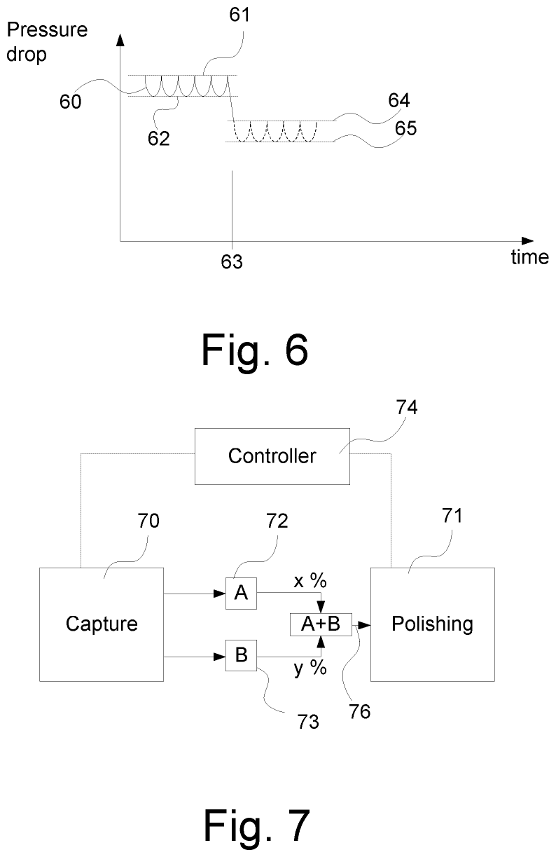

FIG. 6 illustrates the effect of controlling process parameters based on the performance of the raw material used in the bioprocess system. The controller monitors a specific process parameter 60, such as the pressure drop over a chromatography column at constant flow rate, which process parameter 60 varies between a max value 61 and min value 62. The pressure drop is monitored to detect signs of column degradation. An event 63 occurs, such as the column resin is replaced with a new resin lot, and the process parameter is changed to a new level caused by the difference in resin particle size distribution and varies between max value 64 and min value 66. The controller analyses the data properties of the new resin and adjusts the baseline of the pressure monitoring at the new level to allow continued monitoring.

FIG. 7 illustrates in-process treatment, e.g. mixing, between batches, where inherited data properties may be important to be able to adapt the process parameters, using a controller 74, in the following steps while maintaining product quality and/or process performance. In this example, a capture step 70 in a chromatography system produces batches A 72 and B 73, which are mixed before a polishing step 71. In order to qualify the intermediate for further processing, the characteristics of the mixture have to be determined. Some parameters are measured and calculated after mixing, and some are inherited from the batches A and B, similar to what was disclosed in connection with FIGS. 5a and 5b. Even if batch A is not within the predetermined specification, it might be possible to mix it with other batches in order to make the mix fall within the specification. This would reduce scrap and possibly also improve the yield of the system and thus improve the production efficiency.

Illustrative Example

Batch A has a host cell protein concentration of 2000 ng/mL and a target product concentration of 20 mg/mL while Batch B has a host cell protein concentration of 3000 ng/mL and a target product concentration of 10 mg/mL. Host cell protein levels are typically expressed relative to the target product concentration, here Batch A is 2000/20=100 ng/mg and Batch B is 3000/10=300 ng/mg. The subsequent unit operation is validated to purify material with maximum 240 ng/mg of host cell proteins so Batch B isn't possible to use as is. By mixing the two batches it may be possible to qualify the material for further processing.

If one takes 25% of Batch A and 75% of Batch B, the volume weighted average of the relative host cell protein concentration will be 250 ng/mL, calculated by 0.25*100+0.75*300. This value is above the acceptance limit for the subsequent step. However, this is not the correct value for the mixture that instead has to be calculated by estimating the host cell protein concentration and target product concentration separately before determining the relative host cell protein concentration according to:

Host cell protein concentration=0.25*2000+0.75*3000=2750 ng/mL

Target product concentration=0.25*20+0.75*10=12.5 mg/mL

Relative host cell protein concentration=2750/12.5=220 ng/mg which qualifies the mixture for further processing.

The above process described in connection with FIG. 7 may be described as a method in a bioprocess system for processing at least two intermediates, such as batch A 72 and batch B 73, which are a result from a previous process step. The bioprocess system comprises a controller 74 configured to control process parameters of the bioprocess system, and the method comprises a number of steps:
obtaining data properties for each of the at least two intermediates from the previous process step,
performing in-process treatment, such as mixing, of the at least two intermediates 72 and 73 to produce a resulting intermediate 76,
obtaining data properties of the resulting intermediate 76, by measuring and calculating some parameters after treatment and using inherited data properties from the intermediates 72 and 73, and estimating performance of the resulting intermediate 76 when used in a following process step 71,
identifying variations of process parameters of the bioprocess system indicative of the quality of the target product and/or process performance, which may be performed in the controller 74, and
adapting the process parameters of the following process step 71 to compensate for variations of process parameters based data properties of each intermediate 72 and 73 from a previous process step 70 and the data properties of the resulting intermediate 76.

According to some embodiments, the at least two intermediates 72 and 73 are selected to be different batches produced in the previous process step 70.

According to some embodiments, the bioprocess system is selected to be a chromatography system, and the previous process step 70 is a capture step and the following step 71 is a polishing step.

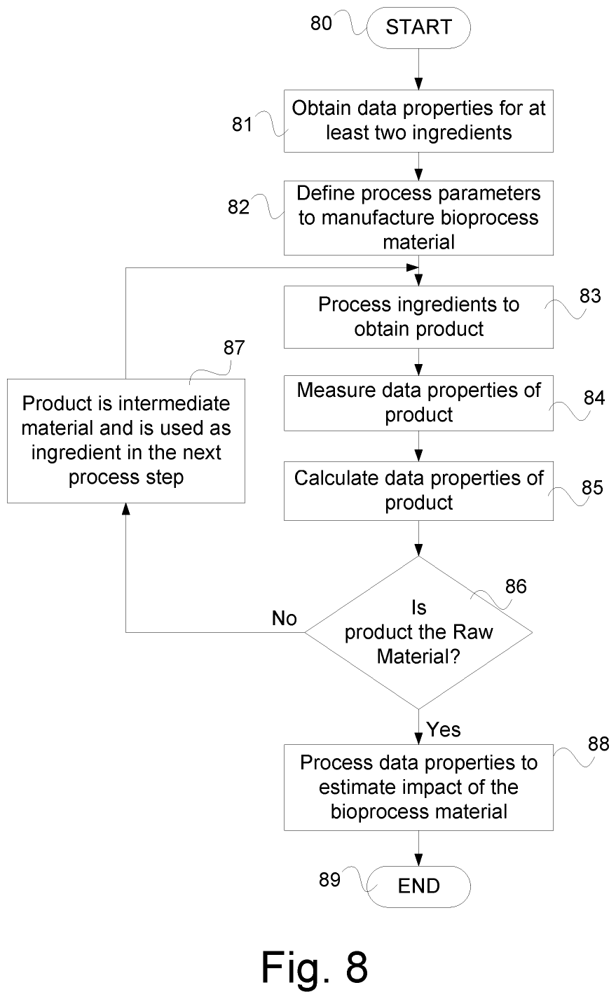

FIG. 8 is a flowchart illustrating a process of estimating performance in a material when used in a bioprocess. The bioprocess material comprises at least two ingredients, each having data properties. According to some embodiments, the bioprocess material is a chromatography resin. According to another embodiment, the bioprocess material is cell culture media.

The process is started in step 80, and in step 81 the data properties for the at least two ingredients used to produce the bioprocess material are obtained. This may be from a CofA (Certificate of Analysis) provided by the manufacturer of the ingredient, or properties are measured and calculated before use.

According to some embodiments, step 81 comprises obtaining for at least one ingredient, such as each ingredient, a particle size distribution. According to some embodiments, each ingredient comprises at least one substance, and process further comprises selecting the data properties of each ingredient to comprise lot number, supplier of the ingredient, and data characterizing the at least one substance. According to some embodiments, the process further comprises, for ingredients with at least two substances, selecting the data properties of each ingredient to further comprise the ratio between the at least two substances.

Other data properties may comprise molecular species representing drug substances such as isoforms.

In step 82, procedures to produce the bioprocess material are defined. The procedures comprise different steps and in some cases also include producing intermediate materials which are used as ingredients in the following process steps, as explained below in step 87.

According to some embodiments, the procedures comprises any combination of the group: filtration, reacting, cooling, activation, mixing, diluting, sieving, washing, grinding and heating.

When the procedures are defined, the flow continues to step 83, in which the at least two ingredients are processed according to the defined procedures to obtain at least one product. The product may be an intermediate material or be the bioprocess material (a.k.a. raw material for the user). In step 84 data properties of each product is measured, and in step 85 data properties of each product is calculated based on the measured data properties of each product and/or data properties from the at least two ingredients.

In some embodiments, step 84 comprises obtaining at least data properties related to, particle size distribution and/or porosity and/or flow rate of each intermediate material.

In step 86 a decision is made regarding the status of the produced product. If the product is the bioprocess material, then the flow continues to step 88, and if the product is not the bioprocess material, then the flow continues to step 87.

In step 88, the measured and calculated data properties are processed to estimate the impact of the bioprocess material on a target product in the bioprocess system. According to some embodiments, step 88 further comprises retrieving information of the manufacturing process of the target product in the bioprocess system, and mapping the estimated performance of the bioprocess material on the manufacturing process to estimate the impact of the bioprocess material on a target product.

In step 87, the product is treated as an intermediate material and steps 83-86 are repeated with each product as one of the at least two ingredients.

Thus, the above described method describes how raw material (i.e. bioprocess material) is manufacture from at least two ingredients. In some embodiments, the raw material is produced via intermediate materials. The result from measurements of data properties and calculations of data properties is stored in a data storage and accessible for the control units used when controlling the bioprocess, e.g. a chromatography or cell culture process.

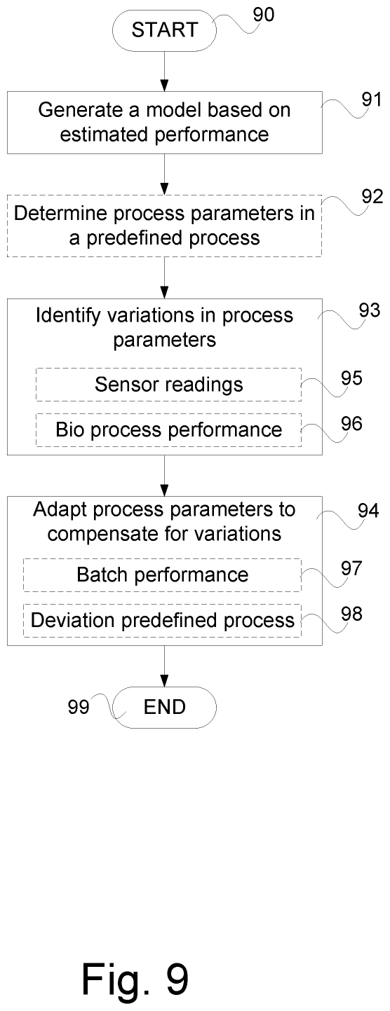

FIG. 9 is a flow chart illustrating a process of adapting process parameters based variations in process parameters in a bioprocess system, e.g. chromatography system, or cell culture system. The bioprocess a controller configured to control process parameters of the chromatography system comprises a controller configured to control process parameters of the bioprocess system.

The flow starts in step 90, and in step 91, a model is generated which is accessible by the controller, based on the estimated performance of the bioprocess material obtained according to a process described in connection with FIG. 8.

The process comprises an optional step 92, in which purification of the target product is performed in a predefined process, and the system is further configured to measure parameter values before and/or after an operation in the bioprocess system. The optional step further comprises identifying deviation between measured parameter values with parameter values obtained in the predefined process and adapting, in step 98, process parameters to compensate for the identified deviation.

The flow continues to step 93, in which variations of process parameters of the bioprocess system is identified, indicative of the quality of the target product and/or process performance.

According to some embodiments, the bioprocess system further comprises at least one sensor configured to measure parameter values, and step 93 further comprises an additional step 95 to obtain sensor readings to identify variations of process parameters. According to some embodiments, step 93 further comprises an additional step 96 of monitoring the bioprocess performance.

The flow continues to step 94, in which the process parameters of the bioprocess system is adapted to compensate for variations of process parameters based on the model.

According to some embodiment, the model is externally generated and/or generated in the controller.

For a chromatography system, the flowchart in FIG. 9 may be described with the bioprocess system being the chromatography system with at least one column with column material adapted for purification of a target product from a feed, the chromatography system further comprising a controller configured to control process parameters of the chromatography system, wherein the method comprising generating, in step 91 a model, accessible by the controller, based on the estimated performance of the column material obtained according to the process described in FIG. 8, identifying, in step 93, variations of process parameters of the chromatography system indicative of the quality of the target product and/or process performance, and adapting in step 94 the process parameters of the chromatography system to compensate for variations of process parameters based on the model.

According to some embodiments, the column material is provided in batches (or lots), each batch/lot having individually estimated performance. The method further comprising adapting, in step 97, the model based on differences in estimated performance between batches/lots.

According to some embodiments, the chromatography system further comprises at least one sensor configured to measure parameter values, and step 93 further comprises obtaining, in step 95, sensor readings to identify variations of process parameters. According to some embodiments, step 93 further comprises monitoring, in step 96, the at least one column performance.

According to some embodiments, the process comprises an optional step 92, wherein purification of the target product is performed in a predefined process, and the system is further configured to measure parameter values before and/or after the at least one column, and the process further comprises identifying deviation between measured parameter values with parameter values obtained in the predefined process and adapting, step 98, process parameters to compensate for the identified deviation.

According to some embodiments, the column material is any of: chromatography resin, membrane, nanofibres, monolith.

For a cell culture system, the flowchart in FIG. 9 may be described with the bioprocess system being the cell culture system comprising a controller configured to feed cell culture media and control process parameters of the cell culture system. The method comprises: generating, in step 91, a model, accessible by the controller based on the estimated performance of the cell culture media obtained according to the process described in connection with FIG. 8, identifying, in step 93, variations of process parameters of the cell culture system indicative of the quality of the cell culture and/or process performance, and, adapting, in step 94, the process parameters of the cell culture system to compensate for variations of process parameters based on the model.

According to some embodiments, the cell culture media is provided in batches (or lots), each batch/lot having individually estimated performance, the method further comprising adapting, in step 97, the model based on differences in estimated performance between batches/lots.

According to some embodiments, the model is externally generated and/or generated in the controller.

The method described above may be implemented in a computer program for controlling process parameters in a bioprocess system, such as a chromatography system or a cell culture system, comprising instructions which, when executed on at least one processor, cause the at least one processor to carry out the method described in connection with FIG. 9. A computer-readable storage medium may be carrying a computer program for controlling process parameters in a bioprocess system.

1. A method for estimating performance of a bioprocess material when used in a bioprocess system, the bioprocess material comprising at least two ingredients, each having data properties, wherein the method comprises:
i) obtaining the data properties for the at least two ingredients used to produce the bioprocess material,
ii) defining procedures to produce the bioprocess material,
iii) processing the at least two ingredients according to the defined procedures to obtain at least one product,
iv) measuring data properties of each product,
v) calculating data properties of each product based on the measured data properties of each product and/or data properties from the at least two ingredients, and
vi) if the product is the bioprocess material, processing the measured and calculated data properties to estimate the impact of the bioprocess material on a target product in the bioprocess system, or
vii) if the product is not the bioprocess material, treating the product as an intermediate material and repeating steps iii)-vi) with each product as one of the at least two ingredients.

2. The method according to claim 1, wherein the data properties of at least one ingredient and/or each product comprise morphological data properties.

3. The method according to claim 1, wherein step i) further comprises obtaining, for at least one ingredient, a particle size distribution.

4. The method according to claim 1, wherein step iv) further comprises obtaining at least data properties related to, particle size distribution and/or porosity and/or flow rate of each intermediate material.

5. The method according to claim 1, wherein the procedures in step ii) further comprises any combination of the group: filtration, reacting, cooling, activation, mixing, diluting, sieving, washing, grinding and heating.

6. The method according to claim 1, wherein at least two of said ingredients are different batches of the same material.

7. The method according to claim 6, wherein said batches differ from each other with respect to one or more data properties.

8. The method according to claim wherein said batches differ from each other with respect to one or more morphological data properties.

9. The method according to claim 1, wherein each ingredient comprises at least one substance, and the method further comprises selecting the data properties of each ingredient to comprise:
lot number
supplier of the ingredient, and
data characterizing the at least one substance

10. The method according to claim 6, wherein the method further comprises, for ingredients with at least two substances, selecting the data properties of each ingredient to further comprise the ratio between the at least two substances.

11. The method according to 1, wherein the bioprocess material is selected to be chromatography resin.

12. The method according to claim 1, wherein the bioprocess material is selected to be cell culture media.

13. The method according to claim 1, wherein step vi) further comprises retrieving information of the manufacturing process of the target product in the bioprocess system, and mapping the estimated performance of the bioprocess material on the manufacturing process to estimate the impact of the bioprocess material on a target product.

14. A method for manufacturing a bioprocess material for a bioprocess, the bioprocess material comprising at least two ingredients, each having data properties, wherein the method comprises:
processing the at least two ingredients using predefined procedures to produce at least one product,
obtaining data properties of each product,
repeatedly processing the at least one product and the at least two ingredients to obtain the bioprocess material, and
identifying data properties of the ingredients and the at least one product relevant for the performance of the bioprocess material.

15. The method according to claim 14, wherein the data properties of at least one ingredient and/or each product comprise morphological data properties.

16. The method according to claim 14, wherein at least two of said ingredients are different batches of the same material.

17. The method according to claim 14, wherein said batches differ from each other with respect to one or more data properties.

18. The method according to claim 17, wherein said batches differ from each other with respect to one or more morphological data properties.

19. A method in a chromatography system with at least one column with column material adapted for purification of a target product from a feed, the chromatography system further comprising a controller configured to control process parameters of the chromatography system, wherein the method comprises:
a) generating a model, accessible by the controller, based on the estimated performance of the column material obtained according to claim 1,
b) identifying variations of process parameters of the chromatography system indicative of the quality of the target product and/or process performance, and
c) adapting the process parameters of the chromatography system to compensate for variations of process parameters based on the model.

20. The method according to claim 19, wherein the column material is provided in batches, each batch having individually estimated performance, the method further comprising adapting the model based on differences in estimated performance between batches.

21. The method according to claim 19, wherein the chromatography system further comprises at least one sensor configured to measure parameter values, and step b) further comprises obtaining sensor readings to identify variations of process parameters.

22. The method according to claim 21, wherein step b) further comprises monitoring the at least one column performance.

23. The method according to claim 21, wherein purification of the target product is performed in a predefined process, and the system is further configured to measure parameter values before and/or after the at least one column, and the method further comprises identifying deviation between measured parameter values with parameter values obtained in the predefined process and adapting process parameters to compensate for the identified deviation.

24. The method according to claim 19, wherein the model is externally generated and/or generated in the controller.

25. The method according to claim 19, further comprising selecting the column material to be any of: chromatography resin, membrane, nanofibres, and/or monolith.

26. A method in a cell culture system generating cell culture to a bioprocess, the cell culture system further comprising a controller configured to feed cell culture media and control process parameters of the cell culture system, wherein the method comprises:
a) generating model, accessible by the controller based on the estimated performance of the cell culture media obtained according to claim 1,
b) identifying variations of process parameters of the cell culture system indicative of the quality of the cell culture and/or process performance, and
c) adapting the process parameters of the cell culture system to compensate for variations of process parameters based on the model.

27. The method according to claim 26, wherein the cell culture media is provided in batches, each batch having individually estimated performance, the method further comprising adapting the model based on differences in estimated performance between batches.

28. The method according to claim 26, wherein the model is externally generated and/or generated in the controller.

29. A method in a bioprocess system for processing at least two intermediates from a previous process step, the bioprocess system comprising a controller configured to control process parameters of the bioprocess system, wherein the method comprising:
obtaining data properties for each of the at least two intermediates from the previous process step,
performing in-process treatment of the at least two intermediates to produce a resulting intermediate,
obtaining data properties of the resulting intermediate, and estimating performance of the resulting intermediate when used in a following process step,
identifying variations of process parameters of the bioprocess system indicative of the quality of the target product and/or process performance, and
adapting the process parameters of the following process step to compensate for variations of process parameters based data properties of each intermediate from a previous process step and the data properties of the resulting intermediate.

30. The method according to claim 29, wherein the in-process treatment comprises mixing of the at least two intermediates.

31. The method according to claim 29, wherein the at least two intermediates are selected to be different batches produced in the previous process step.

32. The method according to claim 29, wherein the bioprocess system is selected to be a chromatography system, and the previous process step is a capture step and the following step is a polishing step.

33. A computer program for controlling process parameters in a bioprocess system, comprising instructions which, when executed on at least one processor, cause the at least one processor to carry out the method according to claim 19.

34. A computer-readable storage medium carrying a computer program for controlling process parameters in a bioprocess system according to claim 33.
